(19) United States
(12) Patent Application Publication
(10) Pub. No.: US 2026/0315251 A1
(43) Pub. Date: Oct. 15, 2026

(54) PROSTHETIC RATCHET SYSTEM

(71) Applicant: Cheryl Welsh, Aiken, SC (US)

(72) Inventor: Cheryl Welsh, Aiken, SC (US)

(21) Appl. No.: 19/645,669

(22) Filed: Apr. 13, 2026

Related U.S. Application Data
(60) Provisional application No. 63/787,056, filed on Apr. 11, 2025.

(51) Int. Cl. A61F 2/78; A61F 2/60; A61F 2/80

(52) U.S. Cl. CPC A61F 2/7812; A61F 2/60; A61F 2/80; A61F 2002/607; A61F 2002/7862; A61F 2002/7875

(57) ABSTRACT

A prosthetic ratchet system is provided. The system is comprised of a prosthetic interface assembly that enhances the stability, comfort, and proprioceptive response of an above-knee prosthesis. The system comprises a custom-molded gel liner configured to conform anatomically to a user's residual limb. The liner further comprises at least one integrated strap aligned with the major axis of tensile force. A prosthetic socket is configured to accommodate the liner and comprises at least one strategically positioned opening through which the strap extends. The strap engages with an external ratcheting mechanism mounted to the socket, wherein the ratcheting mechanism comprises a manual actuation component such as a lever or thumbwheel and a locking mechanism to prevent reverse motion. Upon actuation, the ratcheting mechanism incrementally increases strap tension, thereby creating a compressive preload between the liner and socket and minimizing relative motion during ambulation.

### CROSS-REFERENCE TO RELATED APPLICATION

The present application claims priority to, and the benefit of, U.S. Provisional Application No. 63/787,056, which was filed on Apr. 11, 2025, and is incorporated herein by reference in its entirety.

### FIELD OF THE INVENTION

The present invention relates generally to the field of prosthetics. More specifically, the present invention relates to a prosthetic interface assembly that enhances the stability, comfort, and proprioceptive response of an above-knee prosthesis. Accordingly, the present disclosure makes specific reference thereto. Nonetheless, it is to be appreciated that aspects of the present invention are also equally applicable to other like applications, devices, and methods of manufacture.

### BACKGROUND

Amputees with above-knee limb loss often face persistent challenges in maintaining a secure connection between their residual limb and a prosthetic socket. A common issue involves the prosthetic limb becoming loose during regular use, which can increase the risk of accidental falls and associated injuries. Such instability may result in reduced mobility and physical activity, contributing to a more sedentary lifestyle and negative health outcomes. For many users, repeated detachment or loosening of the prosthesis leads to frustration and dissatisfaction with the device. This dissatisfaction can further discourage prosthesis use, hindering rehabilitation and social participation. Maintaining alignment, preventing axial movement, and ensuring consistent engagement between the limb and prosthesis are key biomechanical requirements that current solutions often fail to meet reliably. Traditional suspension systems, such as suction or pin-lock mechanisms, may not provide sufficient resistance to rotational slippage or accommodate dynamic motion effectively.

Therefore, there exists a long-felt need in the art for a prosthetic ratchet system that prevents loosening or detachment of the prosthesis during daily activities. There also exists a long-felt need in the art for a prosthetic ratchet system that distributes mechanical forces securely while reducing axial and rotational displacement. Moreover, there exists a long-felt need in the art for a prosthetic ratchet system that increases user comfort, safety, and mobility by providing a consistently secure and adjustable interface.

The subject matter disclosed and claimed herein, in one embodiment thereof, comprises a prosthetic ratchet system. The system is comprised of a prosthetic interface assembly that enhances the stability, comfort, and proprioceptive response of an above-knee prosthesis. The system comprises a custom-molded gel liner configured to conform anatomically to a user's residual limb. The liner further comprises at least one integrated strap aligned with the major axis of tensile force. A prosthetic socket is configured to accommodate the liner and comprises at least one strategically positioned opening through which the strap extends. The strap engages with an external ratcheting mechanism mounted to the socket, wherein the ratcheting mechanism comprises a manual actuation component such as a lever or thumbwheel and a locking mechanism to prevent reverse motion. Upon actuation, the ratcheting mechanism incrementally increases strap tension, thereby creating a compressive preload between the liner and socket and minimizing relative motion during ambulation.

In this manner, the prosthetic ratchet system of the present invention accomplishes all the foregoing objectives and provides a prosthetic system that securely attaches a prosthetic limb through an integrated tensioning and locking interface, thereby preventing loosening or detachment. The combination of anatomically contoured liners, reinforced strap systems, and an external ratcheting assembly reduces axial pistoning and rotational slippage while improving proprioceptive feedback. These features collectively address the challenges of user discomfort, instability, and inactivity associated with conventional prosthetic attachment systems. The prosthetic ratchet system ultimately enhances user mobility and safety, enabling amputees to maintain a secure and reliable connection with their prosthetic limbs throughout daily activities.

### SUMMARY

The following presents a simplified summary to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a prosthetic ratchet system. The system is comprised of a prosthetic interface assembly configured to enhance limb stability, user comfort, and proprioceptive response by employing a tension-controlled interface that reduces axial displacement and rotational slippage while promoting synchronized biomechanical motion.

The system is comprised of a custom-molded gel liner shaped to conform to the geometry of a user's residual limb using methods such as casting, digital imaging, or thermoplastic forming. The liner provides anatomical fit, pressure equalization, and dermal protection, and is made from user-specific material selections such as silicone or thermoplastic elastomers. The liner may include regions of variable thickness for optimized pressure distribution and may be configured with a textured surface to enhance frictional engagement with the prosthetic socket.

The liner is further comprised of at least one integrated strap made from low-elongation materials such as aramid fibers or ballistic-grade nylon. The strap is dimensioned and aligned with the major axis of tensile force during use. The strap is fixedly attached to the liner using at least one fastener selected from mechanical joining components. Each fastener is received within a reinforced anchor point or grommet embedded in the liner, which is configured to distribute load and prevent damage under tension.

The system is further comprised of a socket configured as a semi-rigid or rigid shell shaped to accommodate the liner. The socket is fabricated using methods such as lamination or additive manufacturing and is made from materials such as carbon fiber composites or thermoplastic copolyesters. The socket is comprised of at least one opening configured to allow the strap to pass through. The opening is positioned near the distal end of the socket and may be shaped and oriented to align with the strap's attachment vector to reduce shear stress or abrasion.

Upon exiting the socket, the strap engages with a ratcheting mechanism mounted externally on the socket. The ratcheting mechanism is actuated manually through an actuation mechanism such as a lever or rotary dial, which increases strap tension to draw the liner and residual limb into the socket, generating a compressive preload and improving proprioception. The mechanism includes a locking mechanism to prevent the strap from loosening under dynamic loading.

The tensioned strap functions analogously to a biological tendon by providing a continuous mechanical linkage from the socket to the residual limb. The strap transmits external forces and distributes them across the liner without slippage, with the ratcheting mechanism maintaining consistent tension under various conditions.

The invention is also comprised of a method of using the system, including placing the liner on the residual limb, inserting the limb and liner into the socket, passing the strap through the socket opening, engaging the ratcheting mechanism, actuating the mechanism to apply tension, and locking the mechanism to secure the strap during use.

Accordingly, the prosthetic ratchet system of the present invention is particularly advantageous as it provides a prosthetic system that securely attaches a prosthetic limb through an integrated tensioning and locking interface, thereby preventing loosening or detachment. The combination of anatomically contoured liners, reinforced strap systems, and an external ratcheting assembly reduces axial pistoning and rotational slippage while improving proprioceptive feedback. These features collectively address the challenges of user discomfort, instability, and inactivity associated with conventional prosthetic attachment systems. In this manner, the prosthetic ratchet system overcomes the limitations of existing prosthetic systems known in the art and enhances user mobility and safety, enabling amputees to maintain a secure and reliable connection with their prosthetic limbs throughout daily activities.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of a prosthetic ratchet system of the present invention while fully assembled in accordance with the disclosed architecture;

FIG. 2 illustrates a perspective view of a liner of one potential embodiment of a prosthetic ratchet system of the present invention in accordance with the disclosed architecture;

FIG. 3 illustrates a perspective view of one potential embodiment of a prosthetic ratchet system of the present invention with the liner inside the socket and the strap exiting the socket in accordance with the disclosed architecture; and

FIG. 4 illustrates a flowchart of a method of using one potential embodiment of a prosthetic ratchet system of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth to provide a thorough understanding thereof. It may be evident, however, that the innovation can be practiced without these specific details. In other instances, well-known structures and devices are shown in block diagram form to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long-felt need in the art for a prosthetic ratchet system that prevents loosening or detachment of the prosthesis during daily activities. There also exists a long-felt need in the art for a prosthetic ratchet system that distributes mechanical forces securely while reducing axial and rotational displacement. Moreover, there exists a long-felt need in the art for a prosthetic ratchet system that increases user comfort, safety, and mobility by providing a consistently secure and adjustable interface.

The present invention, in one exemplary embodiment, is comprised of a prosthetic ratchet system. The system is comprised of a prosthetic interface assembly that enhances limb stability, user comfort, and proprioceptive response through a tension-controlled interface. This configuration minimizes axial displacement and rotational slippage while facilitating synchronized biomechanical motion.

The system includes a custom-molded gel liner that conforms to the user's residual limb geometry. Shaping methods include casting, digital imaging, or thermoplastic forming. The liner provides anatomical conformity, pressure equalization, and dermal protection, and is fabricated from user-specific materials such as silicone or thermoplastic elastomers. Variable thickness regions are incorporated to optimize pressure distribution, and a textured surface is applied to increase frictional engagement with the prosthetic socket.

The liner incorporates at least one integrated strap constructed from low-elongation materials such as aramid fibers or ballistic-grade nylon. The strap is dimensioned and oriented to align with the principal axis of tensile force. Secure attachment of the strap to the liner is achieved using at least one fastener selected from mechanical joining components. Each fastener is positioned within a reinforced anchor point or grommet embedded in the liner to distribute mechanical load and mitigate damage under tension.

The system further includes a socket configured as a semi-rigid or rigid external shell contoured to fit the liner. Fabrication methods include lamination and additive manufacturing, using materials such as carbon fiber composites or thermoplastic copolyesters. The socket contains at least one opening that permits passage of the strap, with placement near the distal end and geometry tailored to align with the strap's attachment vector, thereby minimizing shear or abrasion.

After passing through the socket, the strap engages a ratcheting mechanism mounted externally. The ratcheting mechanism is manually actuated via an actuation component such as a lever or rotary dial, which progressively increases strap tension. This action draws the liner and residual limb into the socket, generating compressive preload and enhancing proprioceptive feedback. A locking mechanism is incorporated to prevent the strap from loosening under dynamic conditions.

The strap, when tensioned, functions similarly to a biological tendon by establishing a continuous mechanical linkage between the socket and the residual limb. The strap transmits external forces and distributes them across the liner without slippage, while the ratcheting mechanism maintains consistent tension during use.

The invention also includes a method for using the system, which involves applying the liner to the residual limb, inserting both into the socket, routing the strap through the socket opening, engaging the ratcheting mechanism, actuating the mechanism to apply tension, and engaging the locking mechanism to secure the strap.

As a result, the prosthetic ratchet system offers a reliable attachment mechanism by integrating tensioning and locking elements, which prevent loosening or detachment. The system combines anatomically contoured liners, reinforced strap assemblies, and an external ratcheting unit to minimize axial pistoning and rotational slippage while enhancing proprioceptive input. These features collectively address the shortcomings of traditional prosthetic attachments and contribute to improved user comfort, mobility, and safety during daily activities.

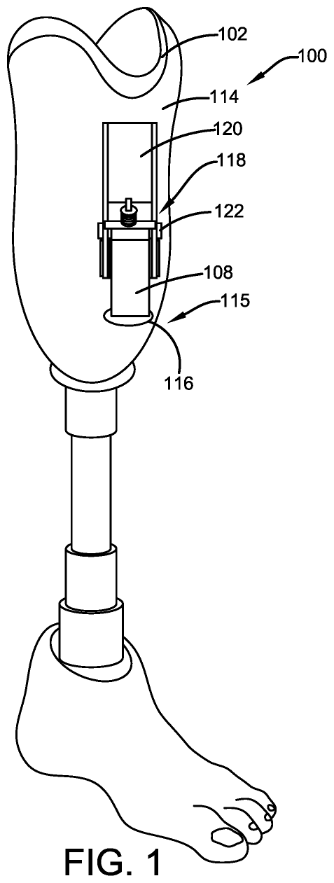

Referring initially to the drawings, FIG. 1 illustrates a perspective view of one potential embodiment of a prosthetic ratchet system 100 of the present invention while fully assembled in accordance with the disclosed architecture. The system 100 may be comprised of a prosthetic interface assembly designed to enhance prosthetic limb stability (of any limb type), user comfort, and proprioceptive response. By incorporating a tension-controlled interface between the residual limb and the external prosthetic socket 114, the system 100 minimizes axial displacement (pistoning) and rotational slippage, while promoting synchronized biomechanical motion during ambulation.

In one embodiment, the system 100 is comprised of a custom-molded gel liner 102. The liner 102 may be shaped via casting, digital imaging, or thermoplastic forming to conform to the unique geometry of a user's residual limb. The liner 102 may provide anatomical fit, pressure equalization, and dermal protection. The liner 102 may be made of materials that may include, but are not limited to, medical-grade silicone, thermoplastic elastomers, urethane-based gels, viscoelastic polymers, etc. Material selection may be based on user-specific factors such as skin sensitivity, perspiration rate, and anticipated load cycles. In one embodiment, the liner 102 may feature variable thickness regions to optimize pressure distribution. For instance, the liner 102 may be configured with increased wall thickness in high-risk areas such as bony prominences, and reduced thickness at flexion zones to preserve range of motion. In a further embodiment, a surface 104 of the liner 102 may be comprised of a texture 106. The texture 106 may be selected to enhance frictional engagement with the socket 114 and may include, but is not limited to, micro-patterned ridges, elastomeric protrusions, or embedded mesh elements. In one embodiment, the texture 106 may be applied by overmolding or surface etching during manufacturing.

The liner 102 is further comprised of at least one integrated strap 108. The strap 108 may be fabricated from inelastic or low-elongation materials, such as, but not limited to, woven aramid fibers, carbon fiber-reinforced polymers, ballistic-grade nylon, high-tenacity polyester webbing, etc. The strap 108 may be dimensioned and oriented to align with the major axis of tensile force while the system 100 is being worn by a user.

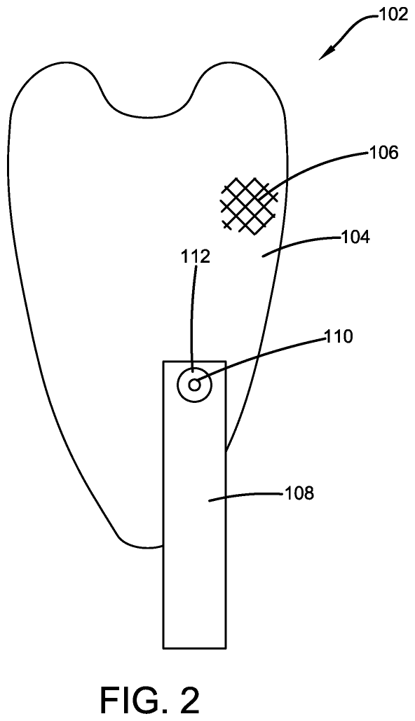

The strap 108 may be fixedly attached to the liner 102 via at least one fastener 110, as seen in FIG. 2. The fastener 110 may be selected from mechanical joining components such as, but not limited to, prosthetic screws, blind rivets, thermoplastic bonding inserts, ultrasonic welds, etc. In one embodiment, the fastener 110 is received within a reinforced anchor point or grommet 112 embedded in the liner 102. The grommet 112 may be comprised of metal, polymer, or composite reinforcement to distribute load and prevent delamination or tearing under tension.

The system 100 is also comprised of a socket 114. The socket 114 may be a semi-rigid or rigid external shell formed to accommodate the outer profile of the liner 102. The socket 114 may be custom-fabricated via lamination, thermoforming, or additive manufacturing. The socket 114 may be made from materials such as, but not limited to, carbon fiber composites, fiberglass-resin laminates, polypropylene, thermoplastic copolyesters, etc. The socket 114 may be any type of prosthetic socket for any type of prosthetic limb/usage.

The socket 114 is comprised of at least one opening 116. The opening 116 may be configured to allow passage of the strap 108 through the socket 114. The opening 116 may be in the form of a linear slit, a radiused channel, a contoured aperture, etc. positioned near the distal end 115 of the socket 114. In various embodiments, the geometry and orientation of the opening 116 may be customized to align with the attachment vector of the strap 108, thereby minimizing shear stress or abrasion.

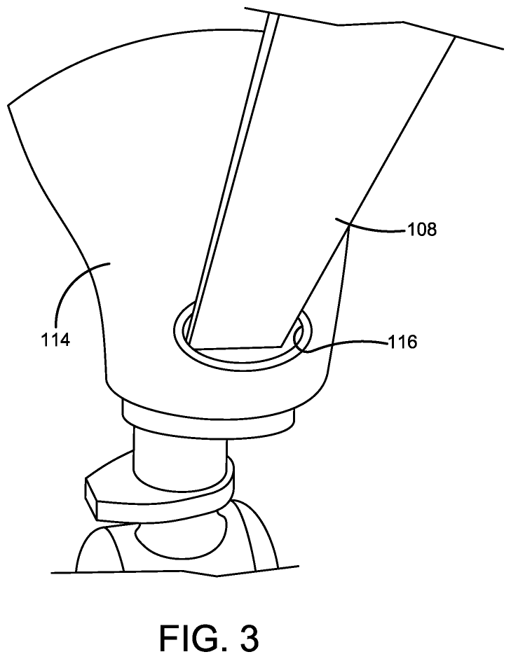

Upon exiting the socket 114 through the opening 116 (as seen in FIG. 3), the strap 108 may engage with a ratcheting mechanism 118, as seen in FIG. 1. The mechanism 118 may be mounted to the external surface of the socket 114 and may be manually or automatically actuated. Manual actuation may be accomplished via an actuation mechanism 120, which may include, but is not limited to, lever-pull mechanisms, thumbwheel dials, rotary knobs, spring-loaded buckles, etc. The ratcheting mechanism 118 may progressively increase tension in the strap 108, thereby drawing the liner 102 and residual limb proximally into the socket 114. This action generates a dynamic compressive preload between the liner 102 and socket 114, eliminating undesired motion and enhancing proprioceptive input. In one embodiment, the mechanism 118 may further be comprised of a locking mechanism 122. The locking mechanism 122 may prevent backdriving of the strap 108 under dynamic loading. Locking mechanisms may include, but are not limited to, one-way cams, spring-loaded detents, pawl-and-ratchet configurations, magnetic locks, etc.

During operation, the tensioned strap 108 may function analogously to a biological tendon, providing a continuous mechanical linkage from the socket 114 to the residual limb. As external forces act upon the prosthesis, tension is transmitted through the strap 108 and distributed across the liner 102, allowing force transfer to occur without slippage or latency. The ratcheting mechanism 118 maintains consistent strap tension and may resist loosening due to material creep, repetitive loading, or environmental conditions.

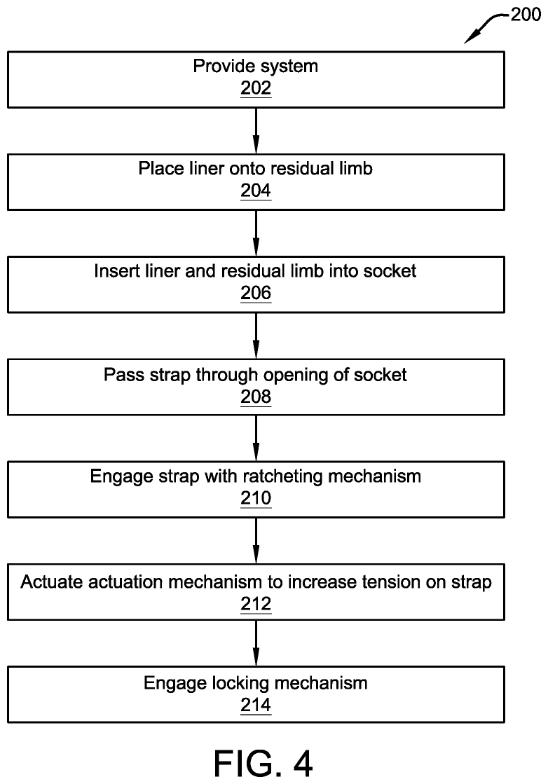

The present invention is also comprised of a method of using 200 the system 100, as seen in FIG. 4. First, a system 100 is provided comprised of a liner 102, at least one integrated strap 108, a socket 114 having at least one opening 116, and a ratcheting mechanism 118 [Step 202]. Then, the liner 102 is placed onto a user's residual limb such that the liner 102 conforms to the anatomical geometry of the limb and the strap 108 extends outwardly from the distal end of the liner 102 [Step 204]. Next, the liner 102 and residual limb are inserted into the socket 114 [Step 206]. The strap 108 is then passed through the opening 116 of the socket 114 [Step 208]. Next, the strap 108 is engaged with the ratcheting mechanism 118 mounted to the external surface of the socket 114 [Step 210]. An actuation mechanism 120 of the mechanism 118 is then actuated manually to progressively increase tension in the strap 108, thereby drawing the liner 102 and residual limb proximally into the socket 114 and establishing compressive preload between the liner 102 and the socket 114 [Step 212]. Finally, a locking mechanism 122 is engaged to prevent backdriving of the strap 108 and to maintain strap tension under dynamic loading conditions [Step 214].

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “prosthetic ratchet system” and “system” are interchangeable and refer to the prosthetic ratchet system 100 of the present invention.

Notwithstanding the foregoing, the prosthetic ratchet system 100 of the present invention and its various components can be of any suitable size and configuration as is known in the art without affecting the overall concept of the invention, provided that they accomplish the above-stated objectives. One of ordinary skill in the art will appreciate that the size, configuration, and material of the prosthetic ratchet system 100 as shown in the FIGS. are for illustrative purposes only, and that many other sizes and shapes of the prosthetic ratchet system 100 are well within the scope of the present disclosure. Although the dimensions of the prosthetic ratchet system 100 are important design parameters for user convenience, the prosthetic ratchet system 100 may be of any size, shape, and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A prosthetic ratchet system comprising:
a liner configured to conform to a residual limb of a user;
a socket configured to receive the liner;
a strap attached to the liner, wherein the strap extends through an opening in the socket; and
a ratcheting mechanism mounted to the socket, the ratcheting mechanism configured to apply a tension to the strap to draw the liner into the socket and establish a compressive preload between the liner and the socket.

2. The prosthetic ratchet system of claim 1, wherein the liner is comprised of a variable thickness.

3. The prosthetic ratchet system of claim 1, wherein the strap is fixedly attached to the liner via a fastener.

4. The prosthetic ratchet system of claim 1, wherein the fastener is comprised of a screw, a rivet, a bonding insert, or an ultrasonic weld.

5. The prosthetic ratchet system of claim 4, wherein the fastener is received within an anchor point embedded in the liner.

6. The prosthetic ratchet system of claim 5, wherein the anchor point is comprised of a grommet.

7. The prosthetic ratchet system of claim 1, wherein the liner is comprised of a medical-grade silicone, a thermoplastic elastomer, a urethane-based gel, or a viscoelastic polymer.

8. A prosthetic ratchet system comprising:
a liner configured to conform to a residual limb of a user, the liner comprised of a texture;
a socket configured to receive the liner;
a strap attached to the liner, wherein the strap extends through an opening in the socket; and
a ratcheting mechanism mounted to the socket, the ratcheting mechanism configured to apply a tension to the strap to draw the liner into the socket and establish a compressive preload between the liner and the socket.

9. The prosthetic ratchet system of claim 8, wherein the texture is comprised of a micro-patterned ridge, an elastomeric protrusion, or an embedded mesh element.

10. The prosthetic ratchet system of claim 8, wherein the opening is comprised of a linear slit, a radiused channel, or a contoured aperture.

11. The prosthetic ratchet system of claim 8, wherein the ratcheting mechanism is comprised of an actuation mechanism.

12. The prosthetic ratchet system of claim 8, wherein the ratcheting mechanism is comprised of a locking.

13. The prosthetic ratchet system of claim 8, wherein the liner is comprised of a variable thickness.

14. The prosthetic ratchet system of claim 8, wherein the strap is fixedly attached to the liner via a fastener.

15. The prosthetic ratchet system of claim 8, wherein the fastener is comprised of a screw, a rivet, a bonding insert, or an ultrasonic weld.

16. The prosthetic ratchet system of claim 14, wherein the fastener is received within an anchor point embedded in the liner.

17. The prosthetic ratchet system of claim 16, wherein the anchor point is comprised of a grommet.

18. The prosthetic ratchet system of claim 8, wherein the liner is comprised of a medical-grade silicone, a thermoplastic elastomer, a urethane-based gel, or a viscoelastic polymer.

19. The prosthetic ratchet system of claim 8, wherein the socket is comprised of a carbon fiber composite, a fiberglass-resin laminate, a polypropylene, or a thermoplastic copolyester.

20. A method of using a prosthetic ratchet system, the method comprising the following steps:
providing a prosthetic ratchet system comprised of comprising a liner, a strap attached to the liner, a socket having an opening, and a ratcheting mechanism;
placing the liner onto a residual limb of a user;
inserting the liner and the residual limb into the socket;
passing the strap through the opening in the socket;
engaging the strap with the ratcheting mechanism; and
actuating an actuation mechanism of the ratcheting mechanism to apply a tension to the strap and draw the liner into the socket to establish a compressive preload.
